(19) United States
(12) Patent Application Publication
(10) Pub. No.: US 2026/0165752 A1
(43) Pub. Date: Jun. 18, 2026

(54) METHOD OF PERFORMING A MINIMALLY INVASIVE SPINAL SURGERY UTILIZING PEDICLE SCREW CORRIDORS

(71) Applicant: ChoSpine, LLC, Englewood Cliffs, NJ (US)

(72) Inventor: Samuel K Cho, Englewood Cliffs, NJ (US)

(73) Assignee: ChoSpine, LLC, Englewood Cliffs, NJ (US)

(21) Appl. No.: 19/418,535

(22) Filed: Dec. 12, 2025

Related U.S. Application Data
(60) Provisional application No. 63/733,009, filed on Dec. 12, 2024.

(51) Int. Cl. A61B 17/70; A61B 1/317; A61B 17/00; A61B 17/02; A61B 17/56; A61B 34/30; A61B 90/00

(52) U.S. Cl. CPC A61B 17/7074; A61B 1/317; A61B 17/00234; A61B 17/025; A61B 17/7001; A61B 34/30; A61B 90/08; A61B 90/37; A61B 2017/00238; A61B 2017/0256; A61B 2017/564; A61B 2090/0807; A61B 2090/373; A61B 2090/376; A61B 2090/378

(57) ABSTRACT

The present disclosure provides a method of performing a minimally invasive spinal surgical procedure comprising making a percutaneous pedicle screw skin incision on the back of a patient over targeted vertebrae for insertion of a pedicle screw therethrough and performing an endoscopic surgical procedure through the percutaneous pedicle screw skin incision. The percutaneous pedicle screw skin incision may be about 0.5-3.5 cm in length. The method may further comprise making a secondary percutaneous pedicle screw skin incision on the back of the patient over the targeted vertebrae. The endoscopic surgical procedure may be a discectomy, laminotomy, laminectomy, foraminotomy, spinal fusion and/or intervertebral fusion. The method allows multiple spinal procedures to be performed through the same small skin incisions typically made for percutaneous pedicle screw fixation, reducing total incisions while maintaining minimally invasive surgical access.

### CROSS-REFERENCE TO RELATED APPLICATIONS

This application claims the benefit of U.S. patent application Ser. No. 63/733,009, filed Dec. 12, 2024, which is hereby incorporated by reference in its entirety for all purposes.

### FIELD OF INVENTION

The present disclosure relates to minimally invasive spinal surgical procedures, and more particularly to a method for performing multiple spinal procedures including decompression and fusion through the same small skin incisions typically used for percutaneous pedicle screw fixation.

### BACKGROUND

Spinal disorders and conditions affecting the vertebral column represent a significant source of pain and disability for millions of patients worldwide. These conditions can arise from various causes including degenerative disc disease, spinal stenosis, spondylolisthesis, herniated discs, and traumatic injuries. Treatment of such spinal pathologies often requires surgical intervention to address neural compression, restore spinal stability, and alleviate patient symptoms.

Traditional open spinal surgery has historically involved large surgical incisions and extensive dissection of surrounding soft tissues including muscles, ligaments, and blood vessels to provide adequate visualization and access to the spinal structures. While effective, these open approaches can result in significant tissue trauma, increased blood loss, prolonged recovery times, and substantial postoperative pain for patients.

The development of minimally invasive spinal surgery techniques has emerged as an alternative approach aimed at reducing surgical morbidity while maintaining therapeutic efficacy. These techniques typically employ smaller skin incisions and specialized instrumentation to minimize disruption of surrounding tissues. Various minimally invasive procedures have been developed for spinal decompression, including discectomy, laminotomy, laminectomy, and foraminotomy, which involve the removal or modification of spinal tissues to relieve neural compression.

Spinal fusion procedures represent another category of spinal surgery that may be performed to stabilize the spine by connecting adjacent vertebrae. These procedures can be accomplished through different approaches including posterior spinal fusion, posterolateral spinal fusion, anterior interbody fusion, lateral interbody fusion, oblique interbody fusion, posterior interbody fusion, and transforaminal interbody fusion. Each approach has particular advantages and limitations depending on the specific pathology being treated.

Percutaneous pedicle screw fixation has become a widely adopted minimally invasive technique for providing spinal stabilization. This approach utilizes small skin incisions, typically measuring only a few centimeters in length, through which pedicle screws can be inserted under fluoroscopic guidance, 3D image guided stereotactic navigation, surgical robotics, virtual, augmented or mixed reality devices, robotically-guided trajectory planning, as well as techniques that utilize electromagnetism, ultrasound, light based and other emerging technologies. The minimally invasive nature of percutaneous pedicle screw placement has made it an attractive option for surgeons seeking to reduce surgical trauma while achieving adequate spinal fixation.

Current minimally invasive spinal surgery practices often involve performing individual procedures through separate surgical approaches and incisions. When multiple spinal procedures are indicated for a single patient, this may result in the creation of additional incisions or the extension of existing incisions to provide adequate surgical access. The ability to perform multiple spinal procedures through a single surgical approach could potentially offer advantages in terms of reduced tissue trauma, decreased operative time, and improved patient outcomes.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an exemplary embodiment of the subject disclosure, a method of performing a minimally invasive spinal surgical procedure is provided. The method comprises making a percutaneous skin incision defined by a trajectory for a pedicle screw on a back of a patient over targeted vertebrae for insertion of a pedicle screw therethrough, and performing an endoscopic surgical procedure through the percutaneous pedicle screw skin incision.

According to aspects of the present disclosure, the method may include one or more of the following features. The percutaneous pedicle screw skin incision may be about 0.5-3.5 cm in length, e.g., about 1-2 cm in length. The method may further comprise making a secondary percutaneous skin incision defined by a trajectory of a pedicle screw on the back of the patient over the targeted vertebrae. The endoscopic surgical procedure is performed using a multi-portal endoscopic technique through the percutaneous pedicle screw skin incision and the secondary percutaneous pedicle screw skin incision. The endoscopic surgical procedure may be a discectomy, a laminotomy, a laminectomy, a foraminotomy, a spinal fusion and/or an intervertebral fusion.

The method may further comprise placing the patient in a prone position on an operating table prior to making the percutaneous skin incisions. The method may further comprise obtaining spinal images before or during the endoscopic surgical procedure. The spinal images are two-dimensional or three-dimensional images obtained using fluoroscopic or stereotactic navigation guidance, surgical robotics, virtual, augmented or mixed reality devices, electromagnetic devices, ultrasound devices, and light based devices. The method may further comprise inserting the pedicle screw through the percutaneous pedicle screw incision after performing the endoscopic surgical procedure. In one aspect, the first percutaneous incision corresponds to a first pedicle receiving an endoscope, and the secondary percutaneous incision corresponds to a second adjacent pedicle receiving a surgical instrument, where working ends of the endoscope and the surgical instrument triangulate at a target spinal pathology between the first and the second pedicles, or vice versa.

According to another aspect of the present disclosure, a method of performing multiple spinal procedures through a single access point is provided. The method comprises creating a small skin incision at a location typical for pedicle screw fixation, performing a decompression procedure through the small skin incision using endoscopic visualization, and performing a spinal fusion procedure through the small skin incision using endoscopic visualization.

According to aspects of the present disclosure, the method may include one or more of the following features. The percutaneous skin incision may be about 0.5-3.5 cm in length. The method may further comprise creating a secondary small skin incision at a location typical for pedicle screw fixation. The decompression procedure and the spinal fusion procedure are performed using a multi-portal endoscopic technique through the small skin incision and the secondary small skin incision. The decompression procedure is selected from the group consisting of a discectomy, a laminotomy, a laminectomy, and a foraminotomy. The spinal fusion procedure comprises decorticating facet joints and placing bone graft material through the small skin incision. Static or expandable interbody cages could also be employed. The method may further comprise completing pedicle screw insertion through the small skin incision.

According to another aspect of the present disclosure, a method of minimally invasive spinal surgery is provided. The method consists essentially of making a percutaneous skin incision of about 0.5-3.5 cm in length on a back of a patient over targeted vertebrae, performing an endoscopic surgical procedure through the percutaneous skin incision, and performing percutaneous pedicle screw insertion through the percutaneous pedicle screw skin incision. The endoscopic surgical procedure is selected from the group consisting of a discectomy, a laminotomy, a laminectomy, a foraminotomy, a spinal fusion, and an intervertebral fusion.

In a further aspect, a trajectory of the decompression is tied to a trajectory of the pedicle screw, where an endoscopic visualization path shares a coaxial or paraxial trajectory with a planned pedicle screw path, thereby using a same muscular corridor. In another aspect, a surgical robot defines a trajectory of the small skin incision, decompression is performed along the robot defined trajectory, and the pedicle screw is inserted by an arm of the surgical robot. In still another aspect, a small skin incision is made on a left pedicle, and decompression is performed on a contralateral (right) side facilitating over-the-top decompression through the same small skin incision. Or, the small skin incision can be made on a right pedicle, and decompression is performed on a contralateral (left) side facilitating over-the-top decompression through the same small skin incision.

In one aspect, the method further comprises a step of making a secondary small skin incision, where the first small skin incision corresponds to a first pedicle receiving an endoscope, and the secondary small incision corresponds to a second adjacent pedicle receiving a surgical instrument, where working ends of the endoscope and the surgical instrument triangulate at a target spinal pathology between the first and the second pedicles, or vice versa. In another aspect, a pedicle screw guidewire can be inserted and left in place during the endoscopic decompression procedure to serve as a retractor or landmark, thereby protecting a nerve root.

### BRIEF DESCRIPTION OF FIGURES

The following detailed description of the exemplary embodiments of the subject disclosure will be better understood when read in conjunction with the appended drawings. For the purpose of illustrating the present disclosure, there is shown in the drawings exemplary embodiments. It should be understood, however, that the subject application is not limited to the precise arrangements and instrumentalities shown.

FIG. 1A is a composite transverse planar view and sagittal planar view of a patient's spine depicting planning for an L4 vertebra pedicle screw incision location using a computed tomography-guided stereotactic navigation system;

FIG. 1B is a composite transverse planar view and sagittal planar view of a patient's spine depicting planning an L5 vertebra pedicle screw incision location using a computed tomography-guided stereotactic navigation system;

FIG. 1C is a view of a patient's back in the vicinity of the lumbar spine with a surgeon marking skin incisions for L4 and L5 pedicle screws that are also to be used for multi-portal endoscopic surgical procedures;

FIG. 2A is a view of a patient's back depicting a surgeon performing multi-portal endoscopic spine surgery using skin incisions made for pedicle screw insertion;

FIG. 2B is a view of a patient's back depicting a surgeon performing a laminotomy using the same skin incisions shown in FIG. 2A;

FIG. 2C is a view of a patient's back depicting a surgeon inserting an intervertebral fusion device and a bone graft using the same skin incisions shown in FIG. 2A;

FIG. 2D is a view of a patient's back depicting a surgeon inserting a pedicle screw through one of the same skin incisions shown in FIG. 2A;

FIG. 3A is a composite transverse planar view and sagittal planar view of a patient's spine similar to FIG. 1A of a surgeon performing decompression (e.g., laminotomy) with a burr through one of the skin incisions shown in FIG. 1C;

FIG. 3B is a composite transverse planar view and sagittal planar view of a patient's spine similar to FIG. 1A of a surgeon performing intervertebral disc removal for interbody fusion through one of the skin incisions shown in FIG. 1C;

FIG. 3C is a composite transverse planar view and sagittal planar view of a patient's spine similar to FIG. 1A of a surgeon inserting an interbody fusion device trial and final implant through one of the skin incisions shown in FIG. 1C;

FIG. 3D is a composite transverse planar view and sagittal planar view of a patient's spine similar to FIG. 1A of a surgeon tapping an L4 vertebra pedicle through one of the skin incisions shown in FIG. 1C; and

FIG. 3E is a composite transverse planar view and sagittal planar view of a patient's spine similar to FIG. 1A of a surgeon inserting an L4 vertebra pedicle screw through one of the skin incisions shown in FIG. 1C.

### DETAILED DESCRIPTION

Reference will now be made in detail to an exemplary embodiment of the subject disclosure illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as upper, lower, top, bottom, above, below and diagonal, are used with respect to the accompanying drawings. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

As used herein, a “pedicle screw corridor” means a trajectory or pathway which a pedicle screw passes through as it is being implanted into bone.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure.

The disclosed methods provide approaches for performing minimally invasive spinal surgical procedures that utilize percutaneous pedicle screw incisions for multiple surgical tasks. In conventional minimally invasive spinal surgery, separate incisions may be made for different procedures, or existing incisions may need to be extended to accommodate additional surgical steps. The methods described herein allow surgeons to perform various spinal procedures through the same small skin incisions that are typically made for percutaneous pedicle screw placement. This approach may reduce the total number of incisions needed and may minimize tissue disruption while maintaining surgical access to target spinal structures.

A method of performing a minimally invasive spinal surgical procedure may involve making percutaneous pedicle screw skin incisions and utilizing these same incisions as surgical corridors for additional procedures. The percutaneous pedicle screw skin incisions may be positioned over targeted vertebrae at locations that provide access for pedicle screw insertion. These incisions may serve dual purposes, functioning both as entry points for pedicle screw placement and as access routes for other surgical instruments and visualization equipment. The size and positioning of the percutaneous pedicle screw skin incisions may be selected to accommodate the range of surgical procedures to be performed while maintaining the minimally invasive nature of the approach.

The method may incorporate endoscopic techniques to enable visualization and surgical manipulation through the small percutaneous incisions. Endoscopic equipment may be introduced through the percutaneous pedicle screw skin incisions to provide visual guidance for surgical procedures that would traditionally require larger exposures. The endoscopic approach may allow surgeons to perform complex spinal procedures while working through incisions that are sized primarily for pedicle screw insertion. Multiple surgical instruments may be introduced through the same incisions in sequence or simultaneously, depending on the specific procedures being performed and the surgical approach selected by the surgeon.

In some cases, the method may involve making multiple percutaneous pedicle screw skin incisions to provide additional access points or to enable multi-portal endoscopic techniques. The number and positioning of incisions may be determined based on the specific vertebral levels being treated and the combination of procedures to be performed. Each incision may serve multiple functions throughout the surgical procedure, transitioning from serving as an access point for one type of procedure to accommodating different instruments or visualization equipment as the surgery progresses. The sequential use of the same incisions for different surgical steps may help maintain the minimally invasive character of the procedure while enabling comprehensive spinal treatment.

The method may involve positioning the patient in a prone position on an operating table to provide access to the posterior aspect of the spine. The prone positioning may allow the surgeon to access the targeted vertebrae through the back of the patient while maintaining anatomical landmarks for accurate incision placement. The operating table may be configured to support the patient in the prone position while allowing for imaging equipment to be positioned around the surgical site. Patient positioning may be adjusted to optimize spinal alignment and provide stable access to the vertebral levels being treated. The prone position may facilitate the placement of percutaneous pedicle screw skin incisions at locations that correspond to the underlying pedicle anatomy.

Spinal imaging may be obtained before the surgical procedure begins to assist with surgical planning and anatomical identification. Two-dimensional spinal images may be acquired using fluoroscopic equipment to provide real-time visualization of the vertebral structures and surrounding anatomy. Three-dimensional spinal images may be obtained using computed tomography or other advanced imaging modalities to create detailed representations of the spinal anatomy at the targeted levels. The imaging data may be used to identify the optimal locations for percutaneous pedicle screw skin incisions and to plan the surgical approach for the various procedures to be performed through these incisions. Pre-surgical imaging may allow the surgeon to assess the anatomical relationships between different spinal structures and to anticipate potential challenges or variations in the surgical approach.

Imaging procedures may also be performed during the surgical procedure to provide ongoing guidance for instrument placement and surgical manipulation. Intraoperative two-dimensional imaging may be used to monitor the progress of surgical procedures and to verify the positioning of instruments relative to anatomical structures. Three-dimensional imaging data may be integrated with navigation systems to provide stereotactic guidance for surgical procedures performed through the percutaneous incisions. The combination of pre-surgical and intraoperative imaging may enable surgeons to perform complex spinal procedures through small incisions while maintaining accuracy and safety. Real-time imaging feedback may allow for adjustments to surgical technique and instrument positioning as the procedure progresses through different phases of treatment.

The imaging preparation may involve calibrating navigation systems and establishing reference points for surgical guidance. Fluoroscopic or computed tomography equipment may be positioned to provide multiple imaging angles while accommodating the endoscopic equipment and surgical instruments being used through the percutaneous incisions. The imaging setup may be configured to minimize interference with the surgical workflow while providing continuous access to anatomical visualization throughout the procedure. Imaging protocols may be established to balance the need for surgical guidance with considerations related to radiation exposure and procedural efficiency. The integration of imaging systems with the surgical approach may enable the performance of multiple spinal procedures through the same small incisions that are typically used for percutaneous pedicle screw placement.

The formation of percutaneous pedicle screw skin incisions may involve creating small openings in the skin that provide access to the underlying spinal structures while maintaining the minimally invasive character of the surgical approach. The percutaneous pedicle screw skin incisions may be made on the back of the patient over the targeted vertebrae at locations that correspond to the anatomical positioning of the pedicles. The size of each percutaneous pedicle screw skin incision may be selected to accommodate the surgical instruments and visualization equipment that will be introduced through the incision during the course of the procedure. The incision dimensions may be tailored to balance the need for adequate surgical access with the goal of minimizing tissue disruption and maintaining small entry points into the surgical site.

The percutaneous pedicle screw skin incisions may be formed with lengths of about 0.5-3.5 cm to provide sufficient access for the various surgical procedures to be performed through the same openings, including without limitation 1.0, 1.5, 2.0, 2.5 and 3.0 cm. The specific length selected for each percutaneous pedicle screw skin incision may depend on factors such as the patient anatomy, the combination of procedures to be performed, and the size of the surgical instruments and endoscopic equipment to be used during the procedure.

The method may involve making a secondary percutaneous pedicle screw skin incision on the back of the patient over the targeted vertebrae to provide additional access points for surgical manipulation and visualization. The secondary percutaneous pedicle screw skin incision may be positioned at a location that complements the primary incision and enables multi-portal endoscopic techniques or multi-instrument approaches. The spacing and orientation of the secondary percutaneous pedicle screw skin incision relative to the primary incision may be determined based on the anatomical relationships of the targeted vertebrae and the specific surgical procedures to be performed. The secondary incision may serve similar functions to the primary incision, accommodating endoscopic equipment, surgical instruments, and providing access for the various spinal procedures that comprise the overall surgical approach.

The surgical approach may follow a sequential methodology where multiple spinal procedures are performed through the percutaneous pedicle screw skin incisions before the final step of pedicle screw insertion. The same small skin incisions that provide access for decompression procedures, fusion techniques, and bone graft placement may subsequently be used for the percutaneous insertion of pedicle screws without the need to create additional skin openings. This sequential approach may allow the surgeon to complete the various components of the spinal procedure while working through a consistent set of access points throughout the surgery. The pedicle screw insertion may be performed as the concluding step of the procedure, utilizing the same percutaneous pedicle screw skin incisions that have been used for the preceding surgical tasks.

The timing of pedicle screw insertion as the final procedural step may allow the surgeon to complete decompression and fusion procedures while maintaining access through the percutaneous incisions for the subsequent hardware placement. The same small skin incisions may accommodate the instruments and guidance systems used for percutaneous pedicle screw insertion after having served as surgical corridors for the earlier phases of the procedure. The sequential use of the percutaneous pedicle screw skin incisions may eliminate the need for additional skin openings specifically for hardware placement, as the existing incisions may provide adequate access for the pedicle screw insertion process. The approach may allow for a comprehensive spinal surgical procedure to be completed through a minimal number of small skin incisions, with each incision serving multiple functions throughout the different phases of the surgical intervention.

The surgical procedures may be performed using various visualization techniques that enable surgeons to work through the small percutaneous pedicle screw skin incisions while maintaining adequate visual guidance for complex spinal manipulations. Direct visualization methods may include observation with the naked eye, which may provide basic visual assessment of anatomical structures that are accessible through the percutaneous incisions. Magnifying glasses may be employed to enhance the visual field and provide improved detail resolution when examining spinal structures through the limited surgical exposure. Surgical loupes may offer additional magnification capabilities and may allow surgeons to perform detailed surgical manipulations while working through the constrained visual access provided by the small incisions. Microscopic visualization may be utilized in cases where high magnification and detailed structural examination are needed for precise surgical procedures performed through the percutaneous access points.

Endoscopic visualization may provide enhanced visual capabilities for surgical procedures performed through the percutaneous pedicle screw skin incisions. The endoscopic equipment may be introduced through the same small incisions used for other surgical instruments, allowing surgeons to visualize internal spinal structures that would not be directly accessible through traditional direct visualization methods. Endoscopic techniques may enable detailed examination of anatomical structures such as neural elements, disc spaces, and bony surfaces while working through incisions that are sized primarily for percutaneous pedicle screw placement. The endoscopic approach may allow surgeons to perform surgical procedures with visual guidance that approaches the detail level available through larger surgical exposures while maintaining the minimally invasive character of the percutaneous approach.

The surgical procedures may be performed without direct visualization by utilizing fluoroscopic, electromagnetic, ultrasound-based, light-based or other guidance systems that provide real-time imaging feedback during surgical manipulation. For example, fluoroscopic navigation may allow surgeons to monitor instrument positioning and surgical progress through continuous radiographic imaging while working through the percutaneous pedicle screw skin incisions. Stereotactic navigation guidance may be employed to provide three-dimensional spatial reference for surgical instruments and anatomical structures during procedures performed through the small incisions. The stereotactic systems may integrate pre-operative imaging data with real-time instrument tracking to enable precise surgical manipulation without relying on direct visual observation of the surgical site. Navigation-guided approaches may allow surgeons to perform complex spinal procedures through percutaneous incisions while maintaining spatial awareness and procedural accuracy through electronic guidance systems such as, e.g., surgical robotics, and virtual, augmented and mixed reality devices.

Hybrid visualization approaches may combine direct visualization techniques with image guidance systems to provide comprehensive visual and spatial information during surgical procedures performed through the percutaneous pedicle screw skin incisions. The combination of direct visualization and image guidance may allow surgeons to utilize both visual observation of accessible anatomical structures and electronic navigation for areas that are not directly visible through the small incisions. Direct visualization methods such as endoscopic observation may be supplemented with fluoroscopic or stereotactic guidance to provide complete spatial awareness during surgical manipulation. The hybrid approach may enable surgeons to transition between different visualization modalities as needed during different phases of the surgical procedure, utilizing the most appropriate guidance method for each specific surgical task performed through the percutaneous access points.

The surgical approach may utilize a multi-portal endoscopic technique that employs separate access points for visualization equipment and surgical instrument insertion. The multi-portal technique may involve using one percutaneous pedicle screw skin incision as a dedicated portal for endoscopic visualization while utilizing a second incision as the primary access point for surgical instrument insertion and manipulation. The separation of visualization and instrument access may allow for improved surgical ergonomics and may provide enhanced visual perspectives during surgical procedures performed through the small incisions. The multi-portal approach may enable surgeons to maintain continuous endoscopic visualization through one portal while introducing various surgical instruments through the separate access point, allowing for coordinated surgical manipulation with sustained visual guidance. The technique may provide improved surgical control and may allow for more complex surgical procedures to be performed through the percutaneous pedicle screw skin incisions while maintaining the minimally invasive character of the overall surgical approach.

Endoscopic surgical procedures may be performed through the percutaneous pedicle screw skin incisions to accomplish various spinal interventions while maintaining the minimally invasive approach. The endoscopic equipment may be introduced through the same small incisions that are positioned for pedicle screw placement, allowing surgeons to perform complex spinal procedures without creating additional surgical access points. The endoscopic approach may enable visualization and manipulation of spinal structures that would traditionally require larger surgical exposures, while working through incisions that are sized primarily for percutaneous hardware insertion. The integration of endoscopic techniques with the percutaneous pedicle screw skin incisions may allow for comprehensive spinal treatment through a minimal number of small access points.

Discectomy procedures may be performed through the percutaneous pedicle screw skin incisions using endoscopic guidance and specialized surgical instruments. The endoscopic equipment may provide visualization of the intervertebral disc space and surrounding neural structures while surgical instruments are introduced through the same incisions to remove disc material. The discectomy may involve the removal of herniated or degenerated disc tissue that may be compressing neural elements, with the surgical manipulation guided by endoscopic visualization through the small percutaneous access points. The endoscopic discectomy approach may allow surgeons to access the disc space and perform tissue removal while working through incisions that maintain the minimally invasive character of the overall surgical procedure. The same percutaneous pedicle screw skin incisions that provide access for the discectomy may subsequently be used for additional surgical procedures and ultimately for pedicle screw insertion.

Laminotomy procedures may be accomplished through the percutaneous pedicle screw skin incisions by introducing endoscopic equipment and bone removal instruments through the same small access points. The laminotomy may involve the partial removal of the lamina to decompress neural structures while preserving spinal stability, with the surgical procedure guided by endoscopic visualization through the percutaneous incisions. Surgical instruments such as drills, burrs, and bone removal tools may be introduced through the percutaneous pedicle screw skin incisions to perform the controlled removal of laminar bone tissue. The endoscopic guidance may allow surgeons to visualize the anatomical relationships between the lamina, neural elements, and surrounding structures while performing the decompression procedure through the small incisions. The laminotomy may be performed as part of a multi-procedure approach that utilizes the same percutaneous access points for subsequent surgical interventions.

Laminectomy procedures may be performed through the percutaneous pedicle screw skin incisions using endoscopic techniques to guide the more extensive removal of laminar bone tissue. The laminectomy may involve the complete removal of the lamina at one or more vertebral levels to provide decompression of neural structures, with the surgical procedure accomplished through the same small incisions used for pedicle screw placement. Endoscopic visualization may enable surgeons to perform the comprehensive bone removal while maintaining visual control over the surgical site through the limited access provided by the percutaneous incisions. The laminectomy procedure may utilize specialized instruments that can be introduced through the small incisions to accomplish the bone removal while working under endoscopic guidance. The same percutaneous pedicle screw skin incisions that provide access for the laminectomy may be used for additional surgical procedures within the same surgical session.

Foraminotomy procedures may be accomplished through the percutaneous pedicle screw skin incisions by utilizing endoscopic guidance to visualize and enlarge the neural foramina. The foraminotomy may involve the removal of bone and soft tissue from the neural foramen to relieve compression of nerve roots, with the surgical procedure performed through the same small incisions that are positioned for pedicle screw insertion. Endoscopic equipment may provide detailed visualization of the foramen and surrounding anatomical structures while surgical instruments are introduced through the percutaneous incisions to perform the decompression. The foraminotomy may utilize drilling, burring, or other bone removal techniques that are guided by endoscopic visualization through the small access points. The procedure may be performed as part of a comprehensive surgical approach that accomplishes multiple spinal interventions through the same percutaneous pedicle screw skin incisions.

Spinal fusion procedures may be performed through the percutaneous pedicle screw skin incisions using endoscopic techniques to guide the preparation and treatment of vertebral surfaces. The spinal fusion may involve the creation of bony connections between adjacent vertebrae to provide spinal stability, with the surgical procedures accomplished through the same small incisions used for pedicle screw placement. Endoscopic visualization may enable surgeons to access and prepare the vertebral surfaces for fusion while working through the limited surgical exposure provided by the percutaneous incisions. The spinal fusion procedures may include posterior fusion techniques that involve the preparation of facet joints and transverse processes for bone graft placement. The same percutaneous pedicle screw skin incisions may accommodate the various instruments and materials needed for the fusion procedures while maintaining the minimally invasive surgical approach.

Intervertebral fusion procedures may be accomplished through the percutaneous pedicle screw skin incisions by utilizing endoscopic guidance to access and prepare the disc space between adjacent vertebrae. The intervertebral fusion may involve the removal of disc material and the preparation of vertebral endplates to create an environment for bony fusion across the disc space. Endoscopic equipment may provide visualization of the intervertebral space while surgical instruments are introduced through the same percutaneous incisions to perform disc removal and endplate preparation. The intervertebral fusion procedure may include the placement of bone graft material, interbody medical devices, and static or expandable interbody cages through the same small incisions that are used for the surgical preparation. The endoscopic approach may allow surgeons to perform comprehensive intervertebral fusion procedures while working through incisions that are sized primarily for percutaneous pedicle screw insertion.

Multiple spinal procedures may be accomplished through the same percutaneous pedicle screw skin incisions in a coordinated surgical approach that addresses various aspects of spinal pathology. The multi-procedure approach may include decompression techniques such as discectomy, laminotomy, laminectomy, and foraminotomy that are performed through the same small incisions using endoscopic guidance. Interbody fusion procedures may be accomplished through the percutaneous incisions with the insertion of medical devices and bone graft material to promote vertebral fusion across the disc space. Posterior and posterolateral fusion techniques may be performed through the same incisions by preparing facet joints and transverse processes for bone graft placement. The comprehensive surgical approach may allow surgeons to address multiple spinal conditions while working through a consistent set of small access points throughout the procedure.

Bone graft material may be inserted through the same percutaneous pedicle screw skin incisions that are used for the various surgical procedures, eliminating the need for separate access points for graft placement. The bone graft may be introduced through the small incisions using specialized instruments that can deliver the graft material to the prepared fusion sites. Endoscopic guidance may assist with the placement of bone graft material by providing visualization of the target areas while the graft is being positioned through the percutaneous incisions. The bone graft insertion may be performed as part of both interbody fusion procedures and posterior fusion techniques, with the same small incisions accommodating the delivery of graft material to multiple anatomical locations. The approach may allow for comprehensive bone graft placement while maintaining the minimally invasive character of the overall surgical procedure.

Facet joint decortication may be performed through the percutaneous pedicle screw skin incisions using endoscopic guidance to visualize and prepare the joint surfaces for fusion. The decortication procedure may involve the removal of the cortical bone surface from the facet joints to expose bleeding bone that may promote fusion with bone graft material. Surgical instruments such as burrs, drills, or other bone preparation tools may be introduced through the same small incisions that are used for other surgical procedures. The endoscopic visualization may enable surgeons to perform controlled decortication of the facet joint surfaces while working through the limited access provided by the percutaneous incisions. The facet joint preparation may be accomplished as part of a posterior fusion approach that utilizes the same percutaneous pedicle screw skin incisions for multiple surgical tasks.

Transverse process decortication may be accomplished through the percutaneous pedicle screw skin incisions by utilizing endoscopic equipment to guide the preparation of these bony surfaces for fusion procedures. The decortication of the transverse processes may involve the removal of cortical bone to create bleeding bone surfaces that may facilitate fusion with bone graft material. Surgical instruments may be introduced through the same small incisions that are used for other spinal procedures to perform the controlled preparation of the transverse process surfaces. The endoscopic approach may provide visualization of the transverse processes and surrounding anatomical structures while the decortication procedure is performed through the percutaneous incisions. The transverse process preparation may be integrated with facet joint decortication and bone graft placement as part of a comprehensive posterolateral fusion approach accomplished through the same small access points.

Interbody medical devices may be inserted through the same percutaneous pedicle screw skin incisions that are used for disc space preparation and other surgical procedures. The interbody devices may include cages, spacers, or other implants that are designed to maintain disc height and provide structural support for intervertebral fusion. Specialized insertion instruments may be used to deliver the interbody medical devices through the small incisions while working under endoscopic guidance. The device insertion may be performed after disc material removal and endplate preparation have been accomplished through the same percutaneous incisions. The interbody medical devices may be positioned within the prepared disc space using endoscopic visualization and may be combined with bone graft material to promote fusion across the intervertebral space. The approach may allow for comprehensive interbody fusion procedures to be accomplished through the same small incisions that are ultimately used for percutaneous pedicle screw insertion.

The integration of multiple surgical procedures through the same percutaneous pedicle screw skin incisions may be accomplished through a coordinated approach that sequences different surgical tasks while maintaining consistent access points throughout the procedure. The method may begin with the formation of small skin incisions at locations that correspond to the anatomical positioning of the pedicles, with these incisions serving as the primary surgical corridors for all subsequent procedures. The sequential performance of decompression procedures, fusion techniques, bone graft placement, and hardware insertion through the same small access points may allow surgeons to accomplish comprehensive spinal treatment while minimizing the total number of incisions and reducing tissue disruption. The coordinated approach may involve transitioning between different surgical instruments and visualization modalities while working through the consistent set of percutaneous access points, allowing each incision to serve multiple functions throughout the different phases of the surgical intervention.

The method performance may involve a systematic progression through various surgical procedures that builds upon the access and visualization capabilities established through the percutaneous pedicle screw skin incisions. The initial phases of the procedure may focus on decompression techniques such as discectomy, laminotomy, laminectomy, or foraminotomy that address neural compression while working through the small incisions with endoscopic guidance. The decompression procedures may be followed by fusion preparation techniques that involve the decortication of facet joints and transverse processes to create bleeding bone surfaces for subsequent bone graft placement. The disc space preparation for interbody fusion may be accomplished through the same incisions by removing disc material and preparing vertebral endplates under endoscopic visualization. The sequential approach may allow each surgical phase to be completed while maintaining the access pathways for subsequent procedures, with the same small incisions accommodating the various instruments and materials needed for each phase of the comprehensive surgical intervention.

The visualization techniques may be integrated throughout the method performance to provide continuous guidance for surgical manipulation while working through the constrained access provided by the percutaneous incisions. Endoscopic equipment may be introduced through the small incisions to provide detailed visualization of internal spinal structures during decompression procedures, fusion preparation, and device placement. The endoscopic visualization may be supplemented with image guidance to provide real-time imaging feedback during instrument positioning and surgical manipulation. Stereotactic navigation systems as well as surgical robotics, and virtual, augmented or mixed reality devices may be integrated with the surgical approach to provide three-dimensional spatial reference for surgical instruments and anatomical structures during procedures performed through the small incisions. The combination of direct endoscopic visualization with image-guided navigation may enable surgeons to perform complex surgical manipulations while maintaining spatial awareness and procedural accuracy through the limited access provided by the percutaneous pedicle screw skin incisions.

The multi-portal endoscopic technique may be integrated into the method performance by utilizing separate percutaneous incisions for visualization and instrument insertion, allowing for improved surgical ergonomics and enhanced visual perspectives during the various surgical procedures. One percutaneous pedicle screw skin incision may serve as a dedicated portal for endoscopic visualization while a second incision can function as the primary access point for surgical instrument insertion and manipulation. The separation of visualization and instrument access may allow surgeons to maintain continuous endoscopic observation through one portal while introducing various surgical instruments through the separate access point for coordinated surgical manipulation. The multi-portal approach may enable more complex surgical procedures to be performed through the small incisions while maintaining sustained visual guidance throughout the different phases of decompression, fusion preparation, and device placement. The technique may provide enhanced surgical control by allowing surgeons to optimize the positioning of both visualization equipment and surgical instruments while working through the percutaneous access points. This method further comprises triangulating the endoscopic visualization device inserted through the cranial pedicle incision and the surgical instrument inserted through the caudal pedicle incision to a convergence point at the interlaminar space or facet joint, or vice versa.

The surgical instruments may be integrated into the method performance through a coordinated approach that introduces different tools through the same percutaneous pedicle screw skin incisions as the surgical procedure progresses through various phases. Decompression instruments such as drills, burrs, and tissue removal tools may be introduced through the small incisions during the initial phases of neural decompression procedures. Fusion preparation instruments may be utilized through the same incisions to perform facet joint decortication, transverse process preparation, and disc space preparation for interbody fusion. Bone graft delivery instruments may be introduced through the percutaneous incisions to place graft material at the prepared fusion sites, including both posterior fusion locations and interbody spaces. Interbody device insertion instruments may be utilized through the same small incisions to position medical devices within the prepared disc spaces under endoscopic guidance. The final phase of the procedure may involve the introduction of pedicle screw insertion instruments through the same percutaneous incisions to complete the hardware placement without creating additional skin openings.

The method integration may involve the coordination of imaging guidance systems with the surgical procedures to provide continuous spatial reference and anatomical visualization throughout the multi-phase surgical approach. Pre-operative imaging data may be integrated with intraoperative navigation systems to provide three-dimensional guidance for surgical procedures performed through the percutaneous incisions. Real-time imaging may be coordinated with the surgical workflow to provide ongoing verification of instrument positioning and surgical progress while working through the small access points. The imaging systems may be configured to accommodate the endoscopic equipment and surgical instruments being used through the percutaneous incisions while providing continuous access to anatomical visualization. The integration of multiple imaging modalities may enable surgeons to transition between different guidance methods as appropriate for each phase of the surgical procedure, utilizing endoscopic visualization for direct observation of accessible structures and electronic navigation for areas that are not directly visible through the small incisions. Note that the trajectory of the decompression is preferably tied to the trajectory of the screw. That is, the endoscopic visualization path shares a coaxial or paraxial trajectory with the planned pedicle screw path. This secures a specific efficiency of the method, where you are not just using the same skin hole, but are using the same muscular corridor.

The comprehensive surgical solution may be achieved through the integration of all procedural elements within a minimally invasive framework that accomplishes multiple spinal interventions through the same small incisions typically used for percutaneous pedicle screw placement. The method may allow surgeons to perform decompression procedures, fusion preparation, bone graft placement, interbody device insertion, and pedicle screw fixation while working through a consistent set of small access points throughout the entire surgical session. The integration of endoscopic visualization, image guidance, specialized surgical instruments, and sequential procedural techniques may enable comprehensive spinal treatment while maintaining the minimally invasive character of the surgical approach. The coordinated method performance may reduce the total number of incisions compared to traditional approaches that may involve separate access points for different surgical procedures, while providing surgeons with the capability to address multiple aspects of spinal pathology through a unified surgical approach that utilizes the same percutaneous pedicle screw skin incisions for all phases of the comprehensive spinal intervention.

Referring to the drawings, there are shown exemplary but non-limitative implementations of the surgical methods and procedures contemplated by the subject disclosure. In this regard, according to an exemplary implementation, a computed tomography (CT)-guided stereotactic navigation system is deployed to plan the incision locations used for both pedicle screw placement and the multi-portal endoscopic surgical procedures in accordance with the subject disclosure.

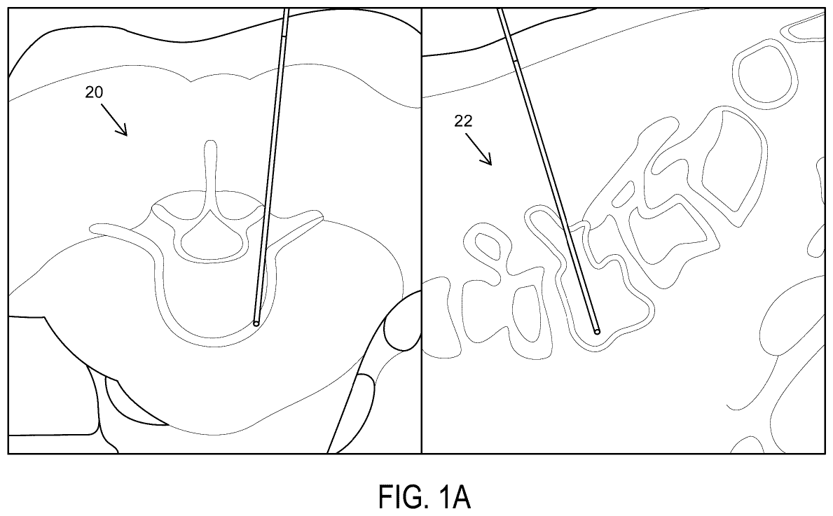

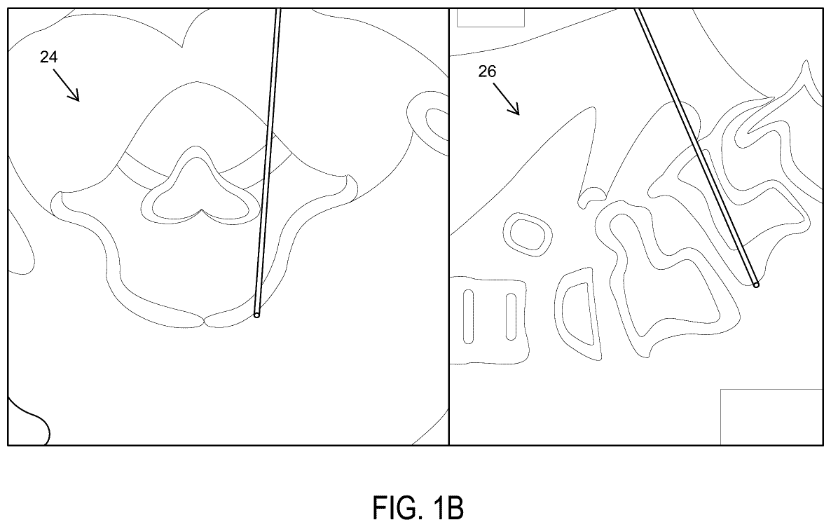

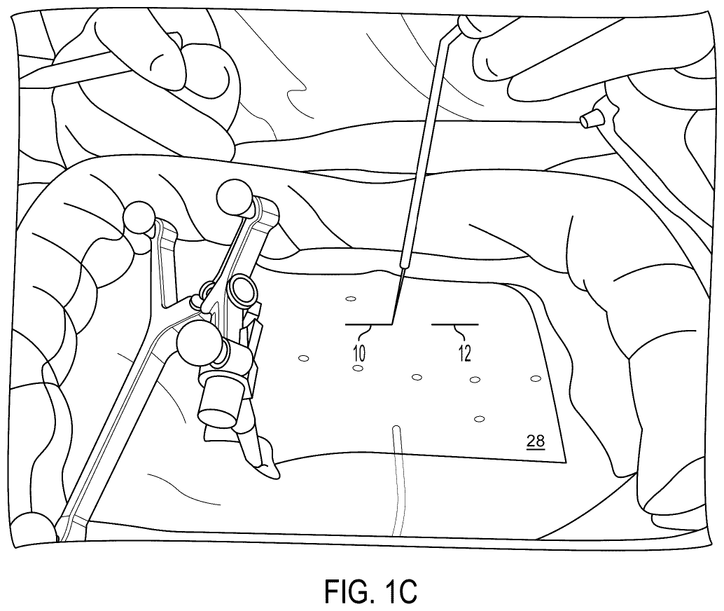

Turning to FIG. 1A, there is shown a composite transverse planar view 20 and sagittal planar view 22 of a patient's spine depicting planning for an L4 vertebra pedicle screw incision location and surgical instrument trajectories using a CT-guided stereotactic navigation system. Similarly, FIG. 1B shows a composite transverse planar view 24 and sagittal planar view 26 of a patient's spine depicting planning an L5 vertebra pedicle screw incision location and surgical instrument trajectories using a CT-guided stereotactic navigation system. It is understood the subject methods and procedures are not limited to surgeries performed on the L4 and L5 vertebrae. Rather, the procedures and methods described herein are applicable to all procedures that may be performed on adjacent vertebrae. Once the optimal incision locations are mapped, the surgeon makes small incisions 10 and 12 into the patient's back 28 as shown in FIG. 1C which correspond to the adjacent vertebrae of interest.

According to the subject disclosure, the incisions 10 and 12 can be from about 0.5 to 3.5 cm in length, including 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9. 3.0, 3.1, 3.2, 3.3 and 3.4 cm.

Further, it is understood that one of the incisions 10, 12 will function as a “viewing portal” and the other the incision will function as a “working portal” during the surgical procedure. That is, incision 10 can be used as the viewing portal and incision 12 can be used as the working portal, or vice versa. Moreover, although not illustrated, additional incisions can be made in the patient's back 28 based on CT or other navigation system mapping when additional vertebrae are involved in the surgical procedure.

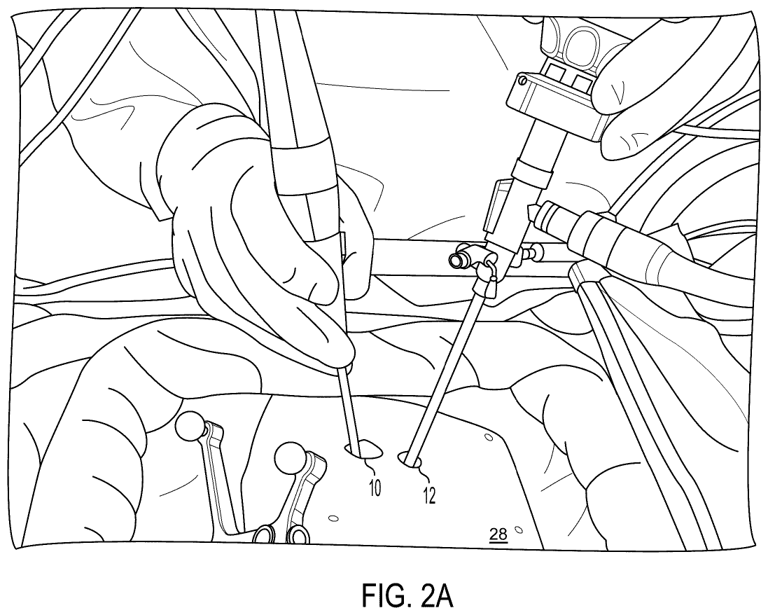

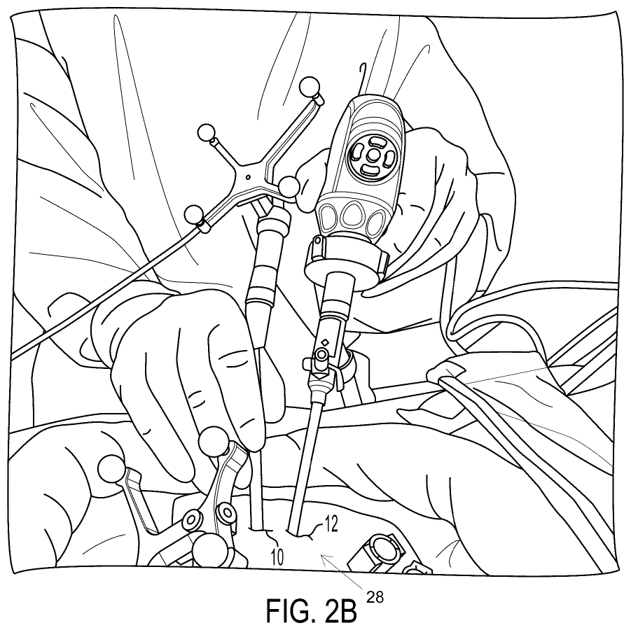

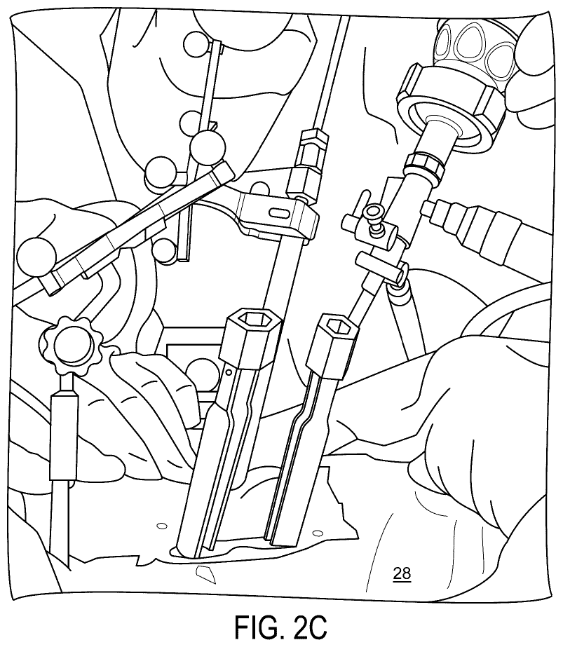

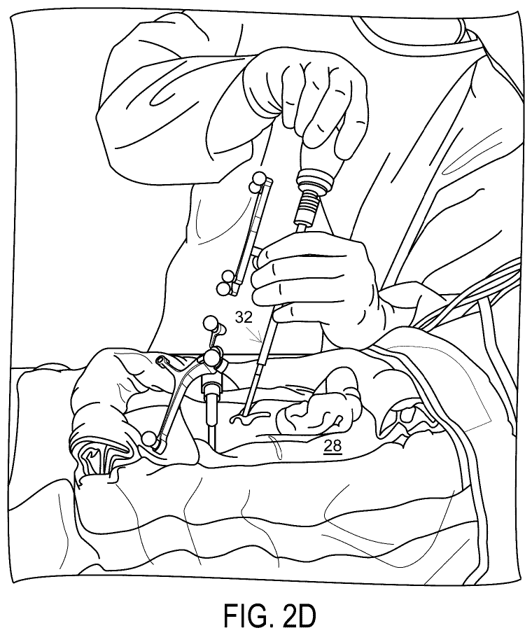

Referring to FIG. 2A, there is shown a view of a patient's back 28 depicting a surgeon performing multi-portal endoscopic spine surgery using skin incisions made for pedicle screw insertion, wherein incision 10 functions as the viewing portal and incision 12 functions as the working portal, understanding that incision 10 can function as the working portal and incision 12 could function as the viewing portal. In one embodiment, a first incision could correspond to a first pedicle (e.g., L4) and receive an endoscope, and a second incision could correspond to a second adjacent pedicle (e. g, L5) and receive a surgical instrument, where the working ends of the endoscope and the instrument triangulate at a target spinal pathology between the first and the second pedicles, or vice versa. In one aspect, the method might comprise triangulating the endoscopic visualization device inserted through a cranial pedicle incision and the surgical instrument inserted through a caudal pedicle incision to a convergence point at the interlaminar space or facet joint, or vice versa.

FIG. 2B is a view of a patient's back 28 depicting a surgeon performing a laminotomy using the same skin incisions shown in FIG. 2A. FIG. 2C is a view of a patient's back 28 depicting a surgeon inserting an intervertebral fusion device 30 and a bone graft using the same skin incisions shown in FIG. 2A. FIG. 2D is a view of a patient's back 28 depicting a surgeon inserting a pedicle screw 32 through one of the same skin incisions shown in FIG. 2A.

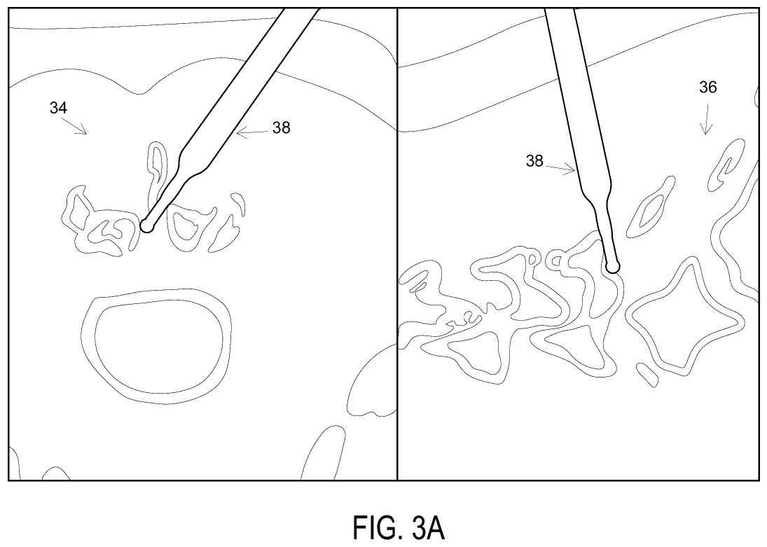

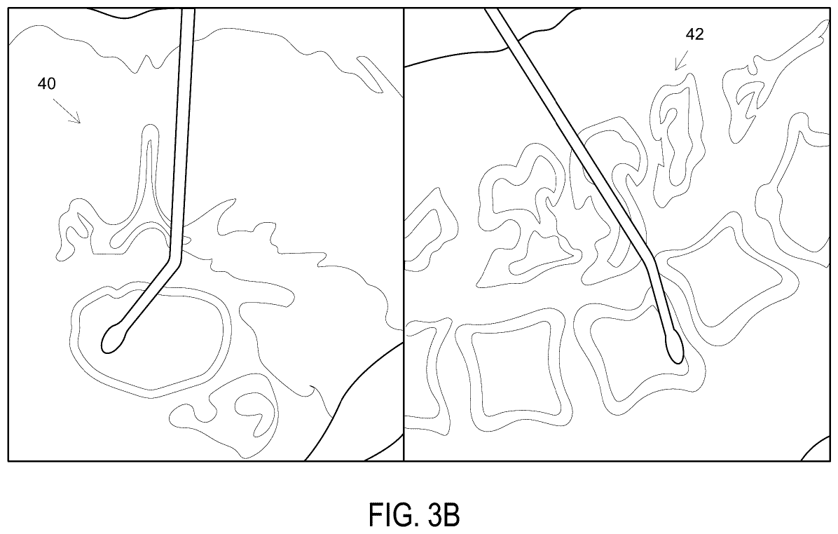

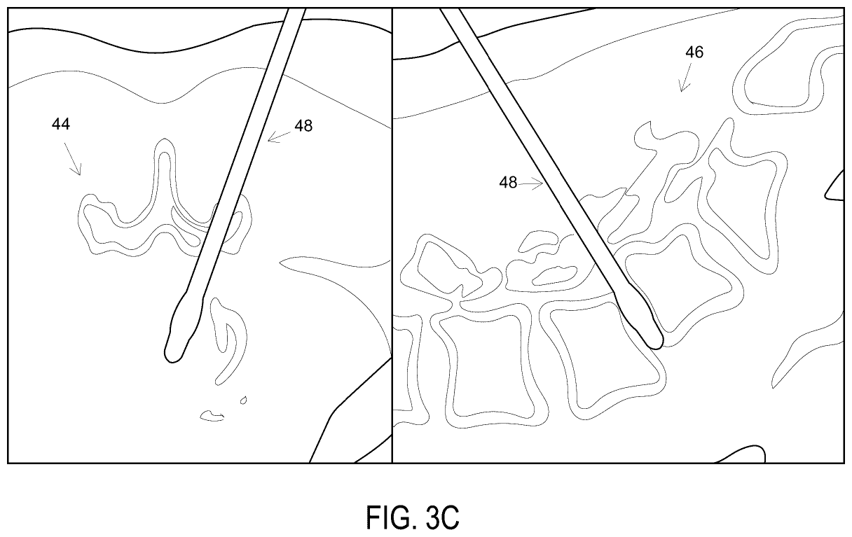

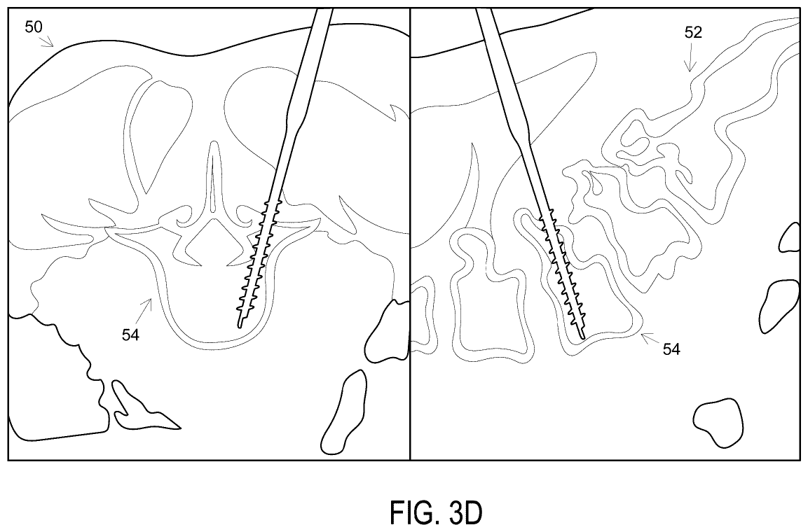

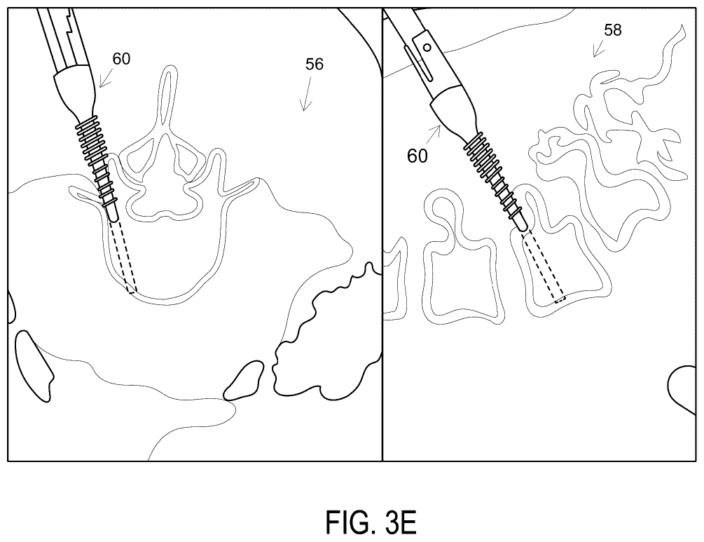

Referring to FIG. 3A, there is shown a composite transverse planar view 34 and sagittal planar view 36 of a patient's spine similar to FIG. 1A showing a surgeon performing decompression (e.g., laminotomy) with a burr 38 through one of the skin incisions shown in FIG. 1C. FIG. 3B is a composite transverse planar view 40 and sagittal planar view 42 of a patient's spine similar to FIG. 1A showing a surgeon performing intervertebral disc removal for interbody fusion through one of the skin incisions shown in FIG. 1C. FIG. 3C is a composite transverse planar view 44 and sagittal planar view 46 of a patient's spine similar to FIG. 1A showing a surgeon inserting an interbody fusion device 48 trial and final implant through one of the skin incisions shown in FIG. 1C. FIG. 3D is a composite transverse planar view 50 and sagittal planar view 52 of a patient's spine similar to FIG. 1A showing a surgeon tapping an L4 vertebra pedicle 54 through one of the skin incisions shown in FIG. 1C. FIG. 3E is a composite transverse planar view 56 and sagittal planar view 58 of a patient's spine similar to FIG. 1A showing a surgeon inserting an L4 vertebra pedicle screw 60 through one of the skin incisions shown in FIG. 1C. Note that the trajectory of the decompression is preferably tied to the trajectory of the screw. That is, the endoscopic visualization path shares a coaxial or paraxial trajectory with the planned pedicle screw path. This secures a specific efficiency of the method, where you are not just using the same skin hole, but are using the same muscular corridor.

In one embodiment, the trajectory of the decompression is preferably tied to the trajectory of the screw. That is, the endoscopic visualization path shares a coaxial or paraxial trajectory with the planned pedicle screw path. This secures a specific efficiency of the method, where you are not just using the same skin hole, but are using the same muscular corridor.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the subject disclosure as defined by the appended claims.

1. A method of performing a minimally invasive spinal surgical procedure, comprising:
making a first percutaneous skin incision defined by a trajectory for a pedicle screw on a back of a patient over a targeted vertebrae; and
performing an endoscopic surgical procedure through the first percutaneous skin incision.

2. The method of claim 1, wherein the first percutaneous skin incision is about 0.5-3.5 cm in length.

3. The method of claim 1, wherein the first percutaneous skin incision is about 1-2 cm in length.

4. The method of claim 1, further comprising:
making a second percutaneous skin incision defined by a trajectory for a pedicle screw on the back of the patient over another targeted vertebrae;
inserting an endoscope through the first percutaneous incision;
inserting a surgical instrument through the second percutaneous skin incision; and
triangulating working ends of the endoscope and the surgical instrument about a targeted spinal pathology of a vertebrae of the patient.

5. The method of claim 4, wherein the endoscopic surgical procedure is performed using a multi-portal endoscopic technique through the first and second percutaneous skin incisions.

6. The method of claim 1, wherein the endoscopic surgical procedure is selected from the group consisting of a discectomy, a laminotomy, a laminectomy, a foraminotomy, a spinal fusion, and an intervertebral fusion.

7. The method of claim 1, wherein the endoscopic surgical procedure comprises removing a portion of a superior articular process or an inferior articular process of a vertebrae.

8. The method of claim 1, further comprising obtaining spinal images before or during the endoscopic surgical procedure.

9. The method of claim 8, wherein the spinal images are two-dimensional or three-dimensional images obtained using fluoroscopic or stereotactic navigation guidance, surgical robotics, virtual, augmented or mixed reality devices, electromagnetic devices, ultrasound devices, and light based devices.

10. The method of claim 1, further comprising inserting a pedicle screw through one of the first and second percutaneous skin incisions after performing the endoscopic surgical procedure.

11. The method of claim 1, further comprising performing a spinal fusion procedure through the first percutaneous skin incision.

12. The method of claim 1, further comprising performing a dissection of underlying soft tissue adjacent the targeted vertebrae through one of the first and second percutaneous skin incisions.

13. A method of performing multiple spinal procedures through a single access point, comprising:
creating a primary skin incision at a location typical for pedicle screw implantation;
performing a decompression procedure through the skin incision using endoscopic visualization; and
performing a spinal fusion procedure through the skin incision using endoscopic visualization.

14. The method of claim 13, further comprising creating a secondary skin incision at a location typical for pedicle screw implantation.

15. The method of claim 14, wherein the decompression procedure and the spinal fusion procedure are performed using a multi-portal endoscopic technique through the skin incision and one of the primary and secondary skin incisions.

16. The method of claim 13, further comprising implanting a pedicle screw insertion through the primary skin incision.

17. The method of claim 16, wherein the decompression procedure is performed through the same trajectory as a trajectory of the pedicle screw, wherein endoscopic visualization is performed through a shared coaxial or paraxial trajectory with the trajectory of the pedicle screw.

18. The method of claim 16, wherein endoscopic visualization is performed through a muscular corridor through which the pedicle screw passes.

19. The method of claim 13, wherein a surgical robot defines a trajectory of the skin incision, decompression is performed along the robot defined trajectory, and the pedicle screw is inserted by an arm of the surgical robot.

20. The method of claim 13, further comprising inserting a guidewire through the primary skin incision and using the guidewire during the decompression procedure as a retractor or landmark.
